(19) United States
(12) Patent Application Publication
(10) Pub. No.: US 2024/0277840 A1
(43) Pub. Date: Aug. 22, 2024

(54) SUPERCONCENTRATED FORMULATIONS OF BIOACTIVE AGENTS

(71) Applicant: UPKARA, INC., Ann Arbor, MI (US)

(72) Inventors: Pravansu Mohanty, Ann Arbor, MI (US); Zhuoran Wang, Ann Arbor, MI (US); Shruti Amle, Ann Arbor, MI (US)

(21) Appl. No.: 18/571,569

(22) PCT Filed: Jul. 1, 2022
(86) PCT No.: PCT/US2022/035906

§ 371 Date: Dec. 18, 2023

Related U.S. Application Data
(60) Provisional application No. 63/217,462, filed on Jul. 1, 2021.

(51) Int. Cl. A61K 39/395; A61K 9/00; A61K 39/00; A61K 47/10; A61K 47/26

(52) U.S. Cl. CPC A61K 39/39591; A61K 9/0019; A61K 47/10; A61K 47/26; A61K 2039/54

(57) ABSTRACT

This disclosure provides super concentrated formulations of one or more bioactive agents in an administration solvent, that may include the bioactive agent being present at a concentration higher than a solution of said bioactive agent in said administration solvent formulated for subcutaneous administration to a subject. The disclosure also provides processes for preparing the super concentrated formulations and processes of treating or preventing disease using the super concentrated formulations.

### CROSS-REFERENCE TO RELATED APPLICATIONS

This application claims priority to U.S. Provisional Patent Application Ser. No. 63/217,462, filed Jul. 1, 2021, which is incorporated by reference herein in its entirety.

### FIELD

The present disclosure concerns formulations of superconcentrated biologically active materials and methods for their preparation.

### BACKGROUND

Delivery of biological agents for the treatment of diseases or conditions is most commonly by intravenous (IV) administration. Biological agents are typically considered therapeutic molecules with high molecular weights (e.g. above 500 Da) and/or are molecules that are similar to those normally found in the body (e.g. antibodies). Monoclonal antibodies (mAbs) make up the majority of the biological agents currently approved for IV administration. Representative examples of mAbs approved for IV infusion are numerous and include Muromonab-CD3 for treatment of organ transplant rejection, infliximab for treatment of rheumatoid arthritis among other conditions, rituximab for treatment of non-Hodgkin's lymphomas, and alemtuzumab for treatment of B-cell chronic lymphocytic leukemia, among a large number of other such mAbs. Other biological agents are also well known that currently require IV administration such as, but not limited to aldesleukin for treatment of melanoma, among others. From a practical perspective, antibody therapy frequently requires large therapeutic doses (e.g., 8 mg/kg for trastuzumab; 375 mg/m² for rituximab), necessitating an injection volume for complete solubilization of the antibody that can only be administered via IV.

One limitation of IV infusion is the potential for rapid clearance of the therapeutic agent. Clearance of biologics from the circulation can occur by either renal filtration or nonspecific binding and uptake (e.g., endothelial pinocytosis). A short circulatory half-life can necessitate frequent injections, from weekly to daily doses, thus increasing the patient's discomfort, overall expense of the treatment regimen, and risks of patient noncompliance.

To increase the plasma half-life, a strategy known as PEGylation has been developed, in which the hydrophilic polymer (polyethylene glycol; “PEG”) is covalently conjugated to the therapeutic. This increases the hydrodynamic size of the therapeutic molecule, thereby reducing its rate of renal clearance and significantly enhancing persistence in the circulation, up to several hundred-fold. However, successful examples of FDA-approved PEGylated biologics for IV administration are limited, e.g., pegloticase (uric acid-metabolizing enzyme) for the treatment of chronic gout.

Other limitations of IV delivery is the potential toxic side effects in non-target organs that can be subjected to high levels of intravenously administered biologic. For example, aldesleukin therapy requires a significantly lower dosage than antibody therapies (0.037 mg/kg per dose), but the systemic infusion of aldesleukin is associated with severe adverse effects that can be potentially fatal, thus requiring inpatient administration. Another recombinant cytokine therapy that has proven extremely difficult to optimize in terms of balancing efficacy with minimal toxicity has been the use of tumor necrosis factor-α (TNF-α) as an immunostimulatory anticancer treatment. TNF-α is believed to act preferentially on tumor endothelium, inducing hyperpermeability that results in hemorrhagic necrosis of the tumor tissue. However, systemic exposure to high levels of TNF-α can cause severe toxicities such as hypotension and septic shock-like syndrome.

The problems of toxicity and rapid clearance after the IV delivery of biologics are both related to the near-immediate systemic availability of the infused therapy. Such rapid availability may be necessary to achieve therapeutic efficacy; however, many biologic drugs benefit from a slower pharmacokinetic distribution through the body (while maintaining systemic availability). In addition, IV infusion is accompanied by the risk of catheter-mediated infection, especially with the emergence of resistant spores in hospital environment, causes significant pain and discomfort, and requires supervised inpatient administration.

For more patient-friendly administration and to enable self-administration, subcutaneous (SC) delivery of biologics is of great interest. A number of licensed biologics are currently administered SC, and the success of this route of administration has been critical for biologic therapies used in managing chronic disease states or symptoms, particularly when coupled with delivery to pre-filled syringes (PFS), pens, or auto-injector devices which allow self- and home-administration. In fact, many PEGylated biologics have been approved for SC injection including the recent COVID-19 vaccines from Pfizer and Moderna. Subcutaneous administration both increases patient compliance and avoids hospitalization, thus reducing treatment costs. Particularly for biologic drugs with short circulatory half-lives that may require frequent dosing, the ability to self-administer therapy via SC injection, as opposed to requiring the continual inpatient administration of IV infusions, provides an enormous benefit to patient quality of life.

Another context in which the use of SC injection is able to improve on IV delivery is in the case of biologic drugs with severe toxicity after systemic infusion. For example, recombinant cytokine therapies such as a and p INFs can provide therapeutic benefits in a number of diseases, including various cancers, hepatitis B and C, and multiple sclerosis, but the IV delivery of such therapies can lead to adverse events after rapid systemic dissemination of the therapeutic. SC administration provides an advantage over IV infusion by creating a relative depot effect at the site of injection: the time required for the therapeutic to drain into systemic circulation effectively prolongs the half-life of the agent in vivo while simultaneously lowering the peak drug level experienced by various compartments, thus reducing the frequency of dosing required as well as reducing the frequency or severity of adverse events.

Although SC injections can be used to achieve a flatter pharmacokinetic profile (a lower peak plasma concentration but a more prolonged duration at an efficacious level), a limitation of the SC route is the injection volume that can be administered without pain, which in humans is typically a maximum of approximately 2 to 2.5 ml of fluid. For regimens of some biologics, such as therapeutic antibodies in oncology applications, the most concentrated form of the drug that can be prepared in a stable, injectable form requires a dosing volume of 5 ml, making traditional SC injection problematic. To overcome this volume limitation, one strategy currently in clinical trials is the use of recombinant human hyaluronidase to locally digest hyaluronic acid, a key polysaccharide component of the extracellular matrix (ECM) in connective tissues.

When hyaluronidase is co-injected with biologics, it generates nanoscale porosity in the local ECM allowing rapid draining of fluid from the injection site and permitting larger volumes of solution to be injected without pain; the matrix self-repairs within about 24 h, making the alterations only transient. Several approved mAbs in oncology (trastuzumab, rituximab) that are currently given as IV infusions are being tested with this modified administration strategy to improve patient compliance with prolonged (>1 year) maintenance therapies that are indicated in conditions such as early breast cancer treatment.

Another motivation for the local injection of biologic drugs is to minimize systemic exposure and the subsequent toxicities that may result. An example of this is the intravitreal injection (directly into the eye) of vascular endothelial growth factor (VEGF) antagonists for the treatment of age-related macular degeneration (AMD). Pegaptinib, an RNA aptamer that targets VEGF, and the anti-VEGF mAbs bevacizumab and ranibizumab have all demonstrated the ability to slow the progression of AMD. However, the systemic infusion of VEGF antagonists can potentially disrupt the normal functions of VEGF in healthy vasculature throughout the body, leading to increased risks of thromboembolic events, hypertension, and impaired wound healing. Early clinical studies showed that intravitreal injections of VEGF antagonists had an improved safety profile compared with IV infusion, with reduced frequency of systemic adverse events.

Another important motivation for the local injection of biologic therapy is to maximize the local concentration of therapy, improving the efficacy of treatment on a specific tissue or organ. For example, the bispecific antibody catumaxomab (anti-CD3/anti-epithelial cell adhesion molecule EPCAM) is injected intraperitoneally for the treatment of malignant ascites resulting from epithelial, gastric, or ovarian cancers. Preclinical pharmacokinetic studies confirmed that intraperitoneal (IP) administration of catumaxomab produced high local concentrations of the antibody in the ascites fluid while significantly limiting systemic exposure (<5% detectable in plasma), a critical finding given the potential toxicity of systemic anti-CD3 stimulation.

To exploit the aforementioned benefits of SC administration, high concentration solutions are needed to keep the injected volume low. High-concentration protein formulations (HCPF) is generally, but imprecisely, applied to preparations ranging between 50 and 150 mg/mL protein, but the physical characteristics of HCPF can be applied to non-protein drugs as well. Characteristics of HCPFs include, for example, increased viscosities, high opalescence, liquid-liquid phase separation, gel formation, or the increased propensity for protein particle formation. Principal goals for the high concentration formulations are protein-stability and good injectability, where the latter requires low-to-moderate viscosity. Mechanisms of chemical degradation include deamidation, oxidation, and iso-asparte formation. Insufficient colloidal stability leads to irreversible aggregation, precipitation and phase separation.

Although limited, commercialized high-concentration biologics, with typical protein concentrations between 150 and 200 mg/mL, but may be lower, are supplied as lyophilized (freeze dried) products. However, for the development of high-concentration, freeze-dried protein formulations, additional challenges appear, such as extremely prolonged reconstitution times and stability issues. Some of the properties observed at high protein concentration impose particular challenges for developing a lyophilized drug product.

Colloidal instability increases at higher protein concentrations. Liquid-liquid phase separation can be enhanced during the freezing step of lyophilization. Phase separation of excipients during lyophilization can also impair protein stability. Excipient phase-separation may be one of the reasons that the “glassy immobilization” concept, often used to explain protein stabilization in lyophilized solids, does not always hold. That is, the protein simply does not “see” the glass. An excipient acting as an effective protein stabilizer not only forms a chemically inert glass, but also “forms a single phase with the protein, which requires a ‘moderate’ interaction with the protein surface to resist separation but yet not denature the protein.” Pikal, M. J. Freeze-drying of proteins. In Stability, Formulation and Delivery of Peptides and Proteins; Cleland, J. L.; Langer, R., Eds., ACS Symposium Series, American Chemical Society: Washington, D.C., 1994; pp 120-133. An excipient that remains hydrogen bonded to the protein during drying cannot be phase-separated from the protein.

The protein's behavior during freezing is another important aspect of lyophilization. At high concentrations (˜50 mg/ml), freezing can increase opalescence accompanied by the formation of visible particles and a decrease in monomer content. Chemical degradation, namely glycation has been associated with freezing process. With increasing protein concentration, the difference between the glass transition and collapse temperature becomes progressively larger. It has been observed that reconstitution times of freeze-dried HCPF are extremely prolonged, up to 30 min and longer.

In summary, high concentration is often a consequence of clinicians' demands for high bioactive agent doses within a limited injection volume. This is not an ideal starting point for developing an efficient freeze-drying cycle. High concentration and high density of solids hinder water-vapor transport and result in longer drying times. There is also the correlation between protein concentration in the lyophilizate and increasing reconstitution time in prior systems.

Thus, new superconcentrated formulations and methods are needed for creating high concentration biologic therapeutic agents to improve efficacy and address needs for patient compliance and comfort.

### SUMMARY

The following summary is provided to facilitate an understanding of some of the innovative features unique to the present disclosure and is not intended to be a full description. A full appreciation of the various aspects of the disclosure can be gained by taking the entire specification, claims, drawings, and abstract as a whole.

Provided are superconcentrated formulations of one or more bioactive agents in an administration solvent. The bioactive agent is present at a concentration higher than a solution of said bioactive agent in said administration solvent formulated for subcutaneous administration to a subject. The administration solvent can be aqueous and can include one or more monovalent or divalent salts. The bioactive agent may be present at a concentration above a concentration that the bioactive agent agglomerates in the solvent. The bioactive agent may be present at a concentration in excess of 50 mg/mL, optionally in excess of 90 mg/mL, optionally in excess of 100 mg/mL, optionally in excess of 150 mg/mL. The bioactive agent may be an antibody, a protein, a lipid particle, optionally a lipid nanoparticle, or other therapeutic agent. The bioactive may be an antibody, optionally a humanized antibody, including muromonab-CD3, infliximab, rituximab, solanezumab, bapineuzumab, catumaxomab, trastuzumab, cetuximab, omalizumab, adalimumab, bevacizumab, BAN2401, tositumomab, or alemtuzumab. The bioactive agent may be a protein, including an interleukin or an interferon, optionally interferon β-1b, Peginterferon alfa-2b, Roferon-A, or aldesleukin. The superconcentrated formulation may further include one or more pharmacologically acceptable excipients. The superconcentrated formulation may be in a volume up to 2.5 mL. The superconcentrated formulation may exclude hyaluronidase. The super concentrated formulation may have a viscosity of 20 cP or less, optionally 12 cP or less, optionally 8 cP or less, optionally 2 cP, or less, optionally 1 cP or less, wherein said viscosity is measured at 20° C. and one atm.

Also provided are processes of preparing a superconcentrated formulation of a bioactive agent in an administration solvent, wherein the process includes overlaying a vitrification mixture comprising the bioactive agent and a vitrification medium on a membrane comprising a capillary network, said membrane in a desiccation chamber; lowering the atmospheric pressure within the desiccation chamber; providing a heat energy to the vitrification mixture, wherein the heat energy is sufficient to prevent the vitrification mixture from entering a cryogenic state during vitrification; desiccating the vitrification mixture by capillary action until the vitrification mixture enters a glassy state; and reconstituting said bioactive agent in an administration solvent, whereby the concentration of said biological agent ns said administration solvent is greater than a concentration of said biological agent in said vitrification medium.

Also provided are processes of treating or preventing a disease or condition including administering to a subject in need a superconcentrated formulation including a bioactive agent and an administration solvent. The bioactive agent is present at a concentration higher than a solution of said bioactive agent in said administration solvent formulated for subcutaneous administration to a subject. The administration solvent can be aqueous and can include one or more monovalent or divalent salts. The bioactive agent may be present at a concentration above a concentration that the bioactive agent agglomerates in the solvent. The bioactive agent may be present at a concentration in excess of 50 mg/mL, optionally in excess of 90 mg/mL, optionally in excess of 100 mg/mL, optionally in excess of 150 mg/mL. The bioactive agent may be an antibody, a protein, a lipid particle, optionally a lipid nanoparticle, or other therapeutic agent. The bioactive may be an antibody, optionally a humanized antibody, including muromonab-CD3, infliximab, rituximab, solanezumab, bapineuzumab, catumaxomab, trastuzumab, cetuximab, omalizumab, adalimumab, bevacizumab, BAN2401, tositumomab, or alemtuzumab. The bioactive agent may be a protein, including an interleukin or an interferon, optionally interferon β-1b, Peginterferon alfa-2b, Roferon-A, or aldesleukin. The superconcentrated formulation may further include one or more pharmacologically acceptable excipients. The superconcentrated formulation may be in a volume up to 2.5 mL. The superconcentrated formulation may exclude hyaluronidase. The super concentrated formulation may have a viscosity of 20 cP or less, optionally 12 cP or less, optionally 8 cP or less, optionally 2 cP, or less, optionally 1 cP or less, wherein said viscosity is measured at 20° C. and one atm.

These and additional features provided by the aspects described herein will be more fully understood in view of the following detailed description, in conjunction with the drawings.

### BRIEF DESCRIPTION OF THE DRAWINGS

The drawings are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present disclosure. Exemplary aspects will become more fully understood from the detailed description and the accompanying drawings, wherein:

FIG. 1 shows an exemplary chamber for capillary assisted vitrification of a vitrification mixture supported by a substrate that includes a plurality of capillary channels according to some aspects as provided herein;

FIG. 2A shows a hydrophilic bed 10 with a thin film of liquid 20 placed a top where the capillary force is significantly higher than the viscous force. This limits the amount of liquid that can be desiccated 21.

FIG. 2B shows a contoured capillary bed, wherein desiccation can preferentially occur at the peaks of the contours 30 where capillary phenomena from the troughs toward the peaks during desiccation can enhance overall vitrification rate and allow for vitrification of large sample volumes relative to FIG. 2A.

FIG. 2C shows liquid filling the surface patterns when there is excess fluid within the contoured capillaries 40, resulting in bubble nucleation and boiling becoming dominant under reduced pressure which may lead to damage of sensitive molecules.

FIG. 3 illustrates the accumulation of vitrified bioactive agent in the capillaries of a capillary membrane according to some aspects by multiple rounds of vitrification.

FIG. 4 illustrates the collection of vitrified material on the fibers of a membrane when formed according to some aspects as provided herein.

FIG. 5 illustrates ELISA studies showing conformational stability and resistance to heat degradation of the vitrified and superconcentrated antibody according to some aspects as provided herein.

FIG. 6 illustrates gel electrophoresis studies showing conformational stability and resistance to heat degradation of the vitrified and superconcentrated antibody according to some aspects as provided herein.

FIG. 7 illustrates XRD analyses of serially vitrified material demonstrating excellent maintenance of amorphous structure following vitrification within the channels of the exemplary membranes according to some aspects as provided herein.

### DETAILED DESCRIPTION

This disclosure provides methods and formulations that address issues with administration of high concentration biological agents that typically are either unable to be administered subcutaneously, or are more effectively administered or desired to be administered at lower administration volumes than was previously achievable. Accordingly, the present disclosure provides an ambient vitrification method for stabilizing high concentrated protein and other therapeutic agent formulations. The absence of freezing, such as is done in prior lyophilization methods, reduces or eliminates phase separation in the formulation and thereby forming a single phase glass matrix. The resulting glass matrix is unexpectedly able to survive reconstitution in solvents suitable for administration to a subject at higher concentrations than could be otherwise achieved thereby creating a system that can be administered at the same total therapeutic dose, but in lower volumes while maintaining therapeutic functionality of the biological agent.

As such, provided are superconcentrated formulations of a biological agent that include a biological agent and an administration solvent, optionally where the administration solvent is suitable for administration to an organism, optionally a human. In some aspects, the biological agent is present in the formulations at a concentration higher than the biological agent is historically used for intravenous administration (if so used) and shows a specific activity (activity per unit mass) with a deviation of less than 10% from prior used solutions when formulated and dosed intravenously or by other traditional method.

The following terms or phrases used herein have the exemplary meanings listed below in connection with at least one aspect.

As used herein, the term “superconcentrated” in some aspects is a solution or suspension of a biological agent at a concentration above the solubility limit of the biological agent in the solvent, wherein the solution is substantially free of aggregates of the biological agent. In the case of a biological agent typically found as a suspension such as a lipid particle, superconcentrated is defined a concentration above which the material may be physically concentrated by evaporation or filtration without the presence of substantial aggregates of the biological agent. In some aspects, “superconcentrated” is defined as a concentration above, which is otherwise achievable in an identical solution/suspension of the biological agent with no observable change in solution colloidal stability, liquid-liquid phase separation or propensity for gel formation. In some aspects, “superconcentrated” is defined as a concentration above which abioactive agent agglomerates in solution such as an administration solvent. In some aspects, a “superconcentrated” solution is one with a thermodynamic stability of the biological agent that is higher than is observed in an otherwise identical solvent.

As used herein “cryogenic” temperature or temperatures for “cryogenesis” or similar refer to a temperature at which a biological sample is cryopreserved, such that biological activity within the biological sample is negligible or absent. It will be understood in some aspects that the cryogenic temperature may include a freezing temperature of the biological sample and/or vitrification medium. It should further be understood that a cryogenic temperature is not bound by a particular threshold or range of values of temperatures in either Fahrenheit or Celsius, but instead can be determined by the relationship between temperature, pressure and molecular energy for the vitrification mixture of interest. It is further to be understood that as used herein, while certainly possible within the definition as set forth, “cryogenesis” and similar derivatives thereof are not limited to temperatures associated with liquid nitrogen at 1 atm or of about −80° C.

“Above cryogenic temperature,” as used herein, accordingly refers to a temperature above the freezing point (Tf) of a vitrification mixture. A point “above cryogenic temperature may further include temperature values where in relation to the surrounding atmosphere and the molecular energy, a cryogenic state is absent. Room temperature, as used herein, refers to a temperature of about 25° C.

As used herein, “boiling” may refer to a point at which a material transitions to a vapor, often marked by the formation of vapor bubbles within the material that can escape into a surround atmosphere and dissipate therein.

“Glass transition temperature” means the temperature above which material behaves like liquid and below which material behaves in a manner similar to that of a solid phase and enters into amorphous/glassy state. This is not a fixed point in temperature, but is instead variable dependent on characteristics of the vitrification mixture of interest. In some aspects, glassy state may refer to the state the vitrification mixture enters upon dropping below its glass transition temperature.

“Amorphous” or “glass” refers to a non-crystalline material in which there is no long-range order of the positions of the atoms referring to an order parameter of 0.3 or less. Solidification of a vitreous solid occurs at the glass transition temperature T_{g}. In some aspects, the vitrification medium may be an amorphous material.

“Crystal” means a three-dimensional atomic, ionic, or molecular structure consisting of one specific orderly geometrical array, periodically repeated and termed lattice or unit cell.

“Crystalline” means that form of a substance that is comprised of constituents arranged in an ordered structure at the atomic level, as opposed to glassy or amorphous. Solidification of a crystalline solid occurs at the crystallization temperature T_{c}.

“Vitrification”, as used herein, is a process of converting a material into an amorphous material. The amorphous solid may be free of any crystalline structure.

“Vitrification mixture” as used herein, means a heterogeneous mixture of biological material(s) and a vitrification medium containing vitrification agents and optionally other materials.

“Biological material” or “biological sample” as used herein, refers to materials that may be isolated or derived from living organisms. Examples of biological materials include, but are not limited to, proteins, cells, tissues, organs, cell-based constructs, or combinations thereof. In some aspects, biological material may refer to mammalian cells. In other aspects, biological material may refer to human mesenchymal stem cells, murine fibroblast cells, blood platelets, bacteria, viruses, mammalian cell membranes, liposomes, enzymes, or combinations thereof. In other aspects, biological material may refer to reproductive cells including sperm cells, spermatocytes, oocytes, ovum, blastocysts, embryos, germinal vesicles, or combinations thereof. In other aspects, biological material may refer to whole blood, red blood cells, white blood cells, platelets, viruses, bacteria, algae, fungi, or combinations thereof.

“Vitrification agent”, as used herein, is a material that forms an amorphous structure, or that suppress the formation of crystals in other material(s), as the mixture of the vitrification agent and other material(s) cools or desiccates. The vitrification agent(s) may also provide osmotic protection or otherwise enable cell survival during dehydration. In some aspects, the vitrification agent(s) may be any water-soluble solution that yields a suitable amorphous structure for storage of biological materials. In other aspects, the vitrification agent may be imbibed within a cell, tissue, or organ.

“Storable or storage,” as used herein, refers to a biological material's ability to be preserved and remain viable for use at a later time.

“Hydrophilic,” as used herein, means attracting or associating preferentially with water molecules. Hydrophilic materials with a special affinity for water maximize contact with water and have smaller contact angles with water relative to hydrophobic materials.

“Hydrophobic,” as used herein, means lacking affinity for water. Materials that are hydrophobic naturally repel water, causing droplets to form, and have large contact angles with water.

“Capillary” as used herein, pertains to or occurring in or as if in a tube of fine bore having a cross sectional area of about 2000 μm² or less.

As used herein, a “subject” is an animal, optionally human, non-human primate, equine, bovine, murine, ovine, porcine, rabbit, or other mammal.

“Cryopreservation” typically refers to rapid cooling of a biological sample, often through the use of liquid nitrogen due to its low temperature, which will rapidly cool a liquid material, or small volume of biological materials by direct immersion. The rate of cooling reduces the mobility of the material's molecules before they can pack into a more thermodynamically favorable crystalline state. Over a more prolonged period, the molecules can arrange to crystallize which can produce damaging results, particularly in biological samples. Water is a significant concern in biological samples as it can crystallize quickly, and its abundance in living tissues can prove to be significantly damaging the more that it is allowed to crystallize. Protective additives, often referred to as cryoprotectants, that interfere with the primary constituent's ability to crystallize may produce amorphous/vitrified material.

A superconcentrated formulation includes one or more bioactive agents. Although not limited, a bioactive agent is optionally one that is made from a living organism or may be made from a living organism. In other aspects, a bioactive agent is one that has functional activity by increasing, decreasing or otherwise altering the concentration, activity, physiological modification, synthesis or degradation of any other compound found in a subject.

Optionally, a bioactive agent is an antibody. The terms “antibody” and “antibodies” as used herein include monoclonal antibodies, polyclonal, chimeric, single chain, bispecific, simianized, and humanized antibodies, drug-antibody conjugates, as well as Fab fragments, including the products of a Fab immunoglobulin expression library. An intact antibody, a fragment thereof (e.g., Fab or F(ab′)₂), or an engineered variant thereof (e.g., sFv) can also be used. Optionally, an antibody is humanized as is recognized in the art. Optionally, an antibody is not humanized. Antibodies can be of any immunoglobulin class including IgG, IgM, IgE, IgA, IgD and any subclass thereof. Illustrative examples of antibodies include antibodies that are bifunctional.

Illustrative non-limiting examples of an antibody include, but are not limited to anti-CD3 mAbs (e.g. otelixizumab, teplizumab, visilizumab), anti-Staphylococcus therapeutic antibodies (e.g. tefibazumab, tosatoxumab, pagibaximab, suvratoxumab, and other mAbs), antibodies for specific cell populations (F4/80—macrophages or c-kit—stem cells), antibodies against injury-associated antigens (VCAM or fibrinopeptide A), or antibodies targeting TGF-β, TNF-α, IL-6, IL-2, CD52, and IL-1β, or combinations of two or more of any of the foregoing, among many others. Other specific illustrative antibodies include, but are not limited to muromonab-CD3, infliximab, rituximab, solanezumab, bapineuzumab, catumaxomab, trastuzumab, cetuximab, omalizumab, adalimumab, infliximab, reticimab, bevacizumab, BAN2401, tositumomab, or alemtuzumab. Such antibodies are commercially available. A bifunctional antibody may include an antibody fusion (either hybridoma fusion or produced chemically) of, as illustrative examples, an anti-CD3 antibody linked to one of the anti-Staphylococcus therapeutic antibodies (e.g. tefibazumab, tosatoxumab, pagibaximab, suvratoxumab and other mAbs), monoclonal antibodies for specific cell populations (F4/80—macrophages or c-kit—stem cells) linked to monoclonal against injury-associated antigens (VCAM or fibrinopeptide A). Methods of making bifunctional antibodies are recognized in the art illustratively as illustrated in Nolan and O'Kennedy, Biochim Biophys Acta., 1990; 1040(1):1-11.

In other aspects, a biological agent is a non-antibody therapeutic. Illustrative examples of such therapeutics include, but are not limited to protein, nucleotide, lipid, or other molecule with biological activity in a subject. More specifically, illustrative examples of non-antibody therapeutic biological agents include, but are not limited to aldesleukin (PROLEUKIN®), interferon α-2a, PEGylated IFN-α-2b, interferon-α-2a (Roferon-A), interferon-α-2b, interferon-β-1b (BETASERON®), insulin glargine, interleukins, abatacept, abobotulinumtoxinA, aflibercept, agalsidase beta, agalsidase beta, albiglutide, albiglutide, alglucosidase alfa, alteplase, cathflo activase, anakinra, asfotase alfa, asparaginase, asparaginase Erwinia chrysanthemi, becaplermin, belatacept, collagenase, collagenase Clostridium histolyticum, darbepoetin alfa, denileukin diftitox, dornase alfa, dulaglutide, ecallantide, elosulfase alfa, epoetin alfa, etanercept, etanercept-szzs, filgrastim, filgrastim-sndz, follitropin alpha, galsulfase, glucarpidase, idursulfase, incobotulinumtoxinA, interferon alfa-2b, interferon alfa-n3, interferon beta-1a, interferon beta-1b, interferon gamma-1b, laronidase, methoxy polyethylene glycol-epoetin beta, metreleptin, ocriplasmin, onabotulinumtoxinA, oprelvekin, palifermin, parathyroid hormone, pegaspargase, pegfilgrastim, pegloticase, rasburicase, reteplase, rilonacept, rimabotulinumtoxinB, romiplostim, sargramostim, sebelipase alfa, tbo-filgrastim, tenecteplase, vedolizumab, ziv-aflibercept, among others.

In other aspects, a biological agent is suitable for use as a vaccine. Illustrative examples of vaccines include BNT162b2, mRNA-1273, JNJ-78436735, influenza virus vaccine (e.g. IIV or LAIV), measles, mumps, and rubella (MMR) vaccine, DTaP vaccine, HepA vaccine, HebB vaccine, Hib vaccine, HPV9 vaccine, MenACWY, PCV13, PPSV23, polio vaccine, rabies vaccine, RV1, RV5, RZV, Vaccinia, VAR, MMRV, yellow fever vaccine, among others.

A formulation includes a bioactive agent present within an administration solvent. An administration solvent is optionally any fluid suitable for use in delivery of a bioactive agent to a subject. Optionally, an administration solvent is an aqueous solvent meaning that the solvent includes water, optionally is predominantly water. Optionally, an administration solvent is water and one or more of a buffer, salt, excipient, or other desired material. An administration solvent optionally is or includes a sugar, optionally dextrose.

An administration solvent optionally includes one or more salts. A salt is optionally a monovalent, divalent, or polyvalent salt. A monovalent salt is optionally a salt of Na, K, Mg, Ca, Zn, or others. Such salts optionally include an anion of chloride, sulfate, phosphate, acetate, or other. Optionally, 1, 2, 3, 4, or more salts are present in the solvent.

An administration solvent optionally includes one or more buffering agents. Illustrative buffering agents may be but are not limited to phosphate, HEPES, TRIS, succinate, citric acid, and acetic acid, among others.

An administration solvent optionally includes an organic (e.g. non-aqueous) predominant or ingredient. Optionally an organic is or includes dimethyl sulfoxide, N-methyl-2-pyrrolidone, glycofurol, Solketal, glycerol formal, acetone, tetrahydrofurfuryl alcohol, diglyme, dimethyl isosorbide, ethyl lactate, among others. Optionally, an administration solvent excludes an organic. Optionally, an organic is present in an administration solvent at a concentration that does not alter the aggregation characteristics of a bioactive agent in the remaining solution absent the organic.

Illustratively, an administration solvent is or includes sodium or phosphate buffered saline.

An administration solvent optionally includes one or more pharmaceutically acceptable excipients. In this context “pharmaceutically acceptable” means an excipient that at the dosage and concentration of the bioactive agent in the formulation, the excipient does not cause any unwanted effects in the subject to who it is administered. Such pharmaceutically acceptable excipients are well-known in the art, and may be illustratively carriers, diluents, binders, disintegrating agents, flow-improving agents, pH-adjusting agents, stabilizing agents, viscosity adjusting agents, preservatives, gelling or swelling agents, surfactants, emulsifying agents, suspending agents, and the like. As recognized by the skilled person, the specific choice of pharmaceutically acceptable excipients depends on the specific form or the formulation, e.g. the dosage form. A person skilled in the art can find guidance in various textbooks, e.g. Remington: The Science and Practice of Pharmacy, in providing suitable pharmaceutically acceptable excipients.

The superconcentrated formulations as provided herein include a bioactive agent present at a concentration that both maintains functional activity of the bioactive agent, but also includes the bioactive agent at a concentration higher than that typically used or achievable by simple evaporation or filtering concentration methods. This for the first time provides a material that may be used for delivering active agents by subcutaneous injection that were historically not able to be tolerated at the volumes required for an adequate does of the bioactive agent, optionally, without the presence of substantial aggregates, viscosity that is too great for subcutaneous delivery apparatuses (e.g. 20 to 30 gauge needles), or significant loss of activity.

In some aspects, the superconcentrated formulations as provided herein are free of or have physiologically irrelevant aggregates of bioactive agent. Physiologically irrelevant aggregates are the absence of aggregates that produce an undesirable immune response to the bioactive agent in a subject or reduce the specific activity of the bioactive agent solution (bioavailability). Free of aggregates is the absence of the presence of at or above a dimer, trimer, tetramer, or other aggregate of 10 or fewer monomers of bioactive agent, or free of an aggregate with a particle size of up to 1 micrometer in diameter as measured by dynamic light scattering.

As such, a formulation as provided herein optionally includes a sufficient amount of bioactive agent that the normal approved dose of the bioactive agent may be administered in a single administration. Dosing of a formulation as provided herein is illustratively by subcutaneous administration as such administration typically has the highest stringency for tolerated volume of the injection. It is appreciated that the formulation may be administered by other routes such as intravenous or intramuscular, or other. Optionally, a formulation as provided herein is formulated to be or is administered subcutaneously, and optionally excludes intravenous and/or intramuscular administration.

As such, a formulation as provided herein optionally has a volume that includes a sufficient amount of bioactive agent for a recommended dose to be administered in a single administration. A formulation is optionally at 5 milliliters (ml) or less, optionally 4 ml or less, optionally 3 ml or less. In some aspects, a formulation is administered at a volume that is tolerated without pain in a human subject, optionally 2.5 ml or less, optionally 2.0 ml or less, optionally 1.5 ml or less.

When a bioactive agent is an antibody or other protein therapeutic, as one example, the bioactive agent is optionally present at a concentration that is at or in excess of 50 milligrams (mg) per ml. Optionally, the concentration of the antibody is at or greater than 75 mg/ml, optionally 90 mg/ml, optionally 100 mg/ml or greater, optionally 125 mg/ml or greater, optionally 150 mg/ml or greater.

A formulation as provided herein has a viscosity, optionally a viscosity greater than normal saline as is used for human administration, optionally at or greater than 1 centipoise (cP). Viscosity as used herein is measured at 20° C. at one atmosphere (i.e. 760 mg/mm). Optionally, the viscosity of the formulation is at or greater than 1.25 cP, optionally at or greater than 1.5 cP, optionally at or greater than 1.75 cP, optionally at or greater than 1.9 cP, optionally at or greater than 2 cP, optionally at or greater than 8 cP, optionally at or greater than 12 cP, optionally at or greater than 20 cP.

A formulation optionally maintains the activity of the bioactive agent substantially to the same level as the agent prior to forming the formulation. The activity of the bioactive agent depends on the identity of the bioactive agent. For example, activity of an antibody is defined as affinity for a target sequence. Activity of an enzyme is defined as ability to react with a substrate. Activity of an immunogen is defined as immunogenic capability. Activity of a nanoparticle, such as a nanoparticle vaccine is optionally ability to enter a target cell. The activity of a bioactive agent in a formulation is optionally at or greater than 75 percent (%) that of the bioactive agent prior to making the formulation. Optionally, the activity of the formulation relative to activity of the bioactive agent prior to making the formulation is 80% or greater, optionally 85% or greater, optionally 90% or greater, optionally 95% or greater, optionally 99% or greater, optionally 99.9% or greater.

The activity of an antibody may be tested and quantified by one of many techniques known in the art. For example, affinity of an antibody may be obtained by enzyme linked immunosorbent assay (ELISA) as is known in the art. Briefly, a sample prior to preparation of the formulation as provided herein as well as a sample of the formulation as provided herein may be tested against a target antigen and the ability of each sample to bind a target antigen that may be bound to the surface of a plate may be measured and directly compared. In other examples, such as when a bioactive agent is a protein such as an peginterferon-α-2a, and one example, may be measured as described by Foser, et al., Protein Expression & Purification, 2002; 30:78-87. Standard activity assays for other protein bioactive agents are also known in the art. Concentrations of a bioactive agent may be measured by function and comparison to a standard curve, by ELISA, or other known methods of quantification.

Also provided are processes of preparation a superconcentrated formulation of a bioactive agent as provided herein. A process includes overlaying a vitrification mixture comprising a biological agent and a vitrification medium on a membrane comprising a capillary network, said membrane in a desiccation chamber; lowering the atmospheric pressure within the desiccation chamber; providing a heat energy to the biological sample, wherein the heat energy is sufficient to prevent the vitrification mixture from entering a cryogenic state; desiccating the vitrification mixture by capillary action until the vitrification mixture enters a glassy state; and reconstituting said biological agent in an administration solvent, whereby the concentration of said biological agent is said administration solvent is greater than a concentration of said biological agent is said vitrification medium.

### Methods of Forming a Formulation

A formulation as provided herein may be made by vitrification of a vitrification mixture of a bioactive agent and a vitrification medium while avoiding cryogenesis and/or crystallization and/or freezing thereof. In certain aspects, vitrification of a bioactive agent with a vitrification medium is performed wherein the vitrification medium includes one or more glass forming agents. In the presence of appropriate glass forming agents, it is possible to store bioactive agents in a vitrified matrix above cryogenic temperatures with vitrification achieved by dehydration. Some animals and numerous plants are capable of surviving complete dehydration. This ability to survive in a dry state (anhydrobiosis) depends on several complex intracellular physiochemical and genetic mechanisms. Among these mechanisms is the intracellular accumulation of sugars (e.g., saccharides, disaccharides, oligosaccharides) that act as a protectant during desiccation, Trehalose is one example of a disaccharide naturally produced in desiccation tolerant organisms.

Sugars like trehalose may offer protection to a bioactive agent in several different ways. A trehalose molecule may effectively replace a hydrogen-bounded water molecule from the surface of a folded protein without changing its conformational geometry and folding due to the unique placement of the hydroxyl groups on a trehalose molecule. A sugar molecule may also prevent leakage from a lipid nanoparticle or a cell during rehydration by binding with the phospholipid heads of the lipid bilayer. Furthermore, many sugars have a high glass transition temperature, allowing them to form an above cryogenic temperature or a room temperature glass at low water content. The highly viscous ‘glassy’ state reduces the molecular mobility, which in turn prevents degradative biochemical reactions that lead to deterioration of cell function and death. Vitrification of biological materials by dehydration in the presence of glass forming sugar trehalose has been disclosed in U.S. Pat. No. 10,433,540 and U.S. Patent Application No. 63/115,936.

A formulation as provided herein may be prepared by a vitrification process that combines low atmospheric pressure and heat energy to achieve even and rapid vitrification of a biological sample in a vitrification mixture. In some aspects, application of heat energy to a vitrification mixture occurs under reduced atmospheric pressure. In some aspects, heat energy is applied to a vitrification mixture to prevent the crystallization of the vitrification mixture.

The temperature of the vitrification mixture is controlled during desiccation and/or vitrification. For example, a vitrification mixture is placed within a desiccation chamber and heat energy is applied to the vitrification mixture to restrict or prevent the vitrification mixture from experiencing a cryogenic temperature. In some aspects, heat energy is transferred to the vitrification mixture to prevent crystallization therein.

Optionally, the temperature of the biological sample is controlled within an applied vacuum or reduction in atmospheric pressure around the vitrification mixture. Application of a low atmospheric pressure can significantly lower the temperature of the vitrification mixture causing the vitrification mixture to crystallize. If crystallization occurs, irrevocable damage can occur to the bioactive agent, which can negatively impact any desired activity or use when reconstituted. Also, reduction in atmospheric pressure around the vitrification mixture can alter the molecular activity within the vitrification mixture, such that the boiling point is reduced. Similar to cryogenesis, boiling the biological sample and/or vitrification medium or overheating can be detrimental. Boiling of a vitrification mixture can lead to bioactive agent loss of tertiary structure, crosslinking and degradation of the components therein, including proteins, fatty acids and nucleic acids and the like, rendering any activity upon reconstitution compromised. In certain aspects, the processes of the present disclosure maintain a vitrification mixture at a temperature above a cryogenic temperature while in low atmospheric pressure such as a vacuum, partial vacuum or in a generally reduced pressure atmosphere.

In certain aspects, the vitrification mixture including the bioactive agent and the vitrification medium may be heated directly to control the temperature of such during desiccation. In other aspects, the vitrification mixture including the bioactive agent and the vitrification medium may have the temperature of such controlled by conduction, convection and/or radiation means. Optionally, the vitrification mixture including the biological sample and the vitrification medium may have its temperature controlled by controlling the temperature outside of the desiccation chamber and relying on conduction through the desiccation chamber or portion thereof to control the temperature of the vitrification mixture. In such instances, it will be appreciated that the physical properties of the walls of the desiccation chamber will need to be taken into consideration. For example, a poorly conducting material of the desiccation chamber may require an applied temperature different from that required by the vitrification mixture in order to allow for the vitrification mixture to receive the appropriate heat energy. Such necessary adaptations will be readily appreciated by those in the art. In some aspects, heat may be applied through a heating pad, a heated bath, a flame, a heated bed, such as glass bead, a heated block and similar. In some cases, the heat energy may be from an electric source of generated heat and/or a heat energy released by combustion and/or a heat energy generated by electrical resistance.

In some aspects, heat energy can be provided to the vitrification mixture through an underlying support substrate. While a porous material of a continuous capillary network may also provide heat energy to the vitrification mixture, in some instances the porous material is of a poor conducting material, such as glass or a polymer. However, the underlying substrate may be of a metal or similarly efficient conducting material and easily connected to a heat source outside of the desiccation chamber or an electrical source and provide heat by resistance created therein. The application of heat energy from the solid support may further provide a temperature gradient to assist in capillary evaporation.

In some aspects, the vitrification mixture including the bioactive agent and the vitrification medium is maintained at a temperature above its cryogenic temperature during vitrification under low atmospheric pressure. Optionally, the vitrification mixture is preheated prior to desiccation under low atmospheric pressure. In other aspects, the vitrification mixture is heated during vitrification under low atmospheric pressure. In other aspects, heat is applied at or around the time vitrification commences. It will be appreciated that the amount of heat energy applied to the vitrification mixture may be constant or may vary during vitrification under low atmospheric pressure process. In some aspects, the introduction of low atmospheric pressure within the desiccation chamber can cause a rapid drop in temperature of the vitrification mixture. In such aspects, having the vitrification mixture ready to receive or already receiving heat energy can increase the recovery rate from the drop in temperature.

In certain aspects, a constant temperature is applied to the vitrification mixture, such that the vitrification mixture is maintained at a temperature of from about T_{g} of the vitrification mixture in ° C. to about 40° C., including about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, and 39° C. In certain aspects, a higher temperature may be applied to the desiccation chamber or the porous material to provide the necessary heat energy to the vitrification mixture. Such applied temperatures may be of from about 15° C. to about 70° C., depending on the size of the desiccation chamber and the conductive means available to transfer effectively to the biological sample and/or vitrification medium.

In some aspects, vitrification of abiological sample is performed at low atmospheric pressure defined as below one atmosphere (760 mmHg). Optionally, the desiccation is performed in a desiccation chamber whereby the vitrification mixture may be placed therein so as to be exposed to low atmospheric pressure. Such a desiccation chamber may be connected to a vacuum source to apply a low atmospheric pressure to the vitrification mixture. A vitrification mixture can be prepared with a vitrification medium or a cryopreservative such as trehalose and subjected to low atmospheric pressure, such as through application of a vacuum. Optionally, the low atmospheric pressure is of from about 0.9 atm to about 0.005 atm, including 0.85, 0.8, 0.75, 0.7, 0.65, 0.6, 0.55, 0.5, 0.45, 0.4, 0.35, 0.3, 0.255, 0.25, 0245, 0.24, 0.235, 0.23, 0.225, 0.22, 0.215, 0.21, 0.205, 0.2, 0.195, 0.19, 0.185, 0.18, 0.175, 0.17, 0.165, 0.16, 0.155, 0.15, 0.145, 0.14, 0.135, 0.13, 0.125, 0.12, 0.115, 0.11, 0.105, 0.1, 0.095, 0.09, 0.085, 0.08, 0.075, 0.07, 0.065, 0.06, 0.055, 0.05, 0.045, 0.04, 0.035, 0.03, 0.025, 0.02, 0.015, and 0.01 atm.

In some aspects, the pressure within the desiccation chamber is lowered to a point above the triple point of the vitrification mixture. In other aspects, the pressure is lowered to a point above the triple point of water, such as greater than 0.006 atm. As set forth herein, lowered atmospheric pressure lowers the temperature of the vitrification mixture while also reducing its boiling point. In some aspects, the pressure within the desiccation chamber is lowered to about 0.04 atm or about 29 mmHg.

The vitrification mixture may be placed in a vacuum or partial vacuum at an elevated temperature or maintained at a temperature above the cryogenic temperature of the vitrification mixture at the atmospheric pressure applied, such that the vitrification mixture does not experience cryogenic temperature during the rapid decrease in atmospheric pressure. During vitrification, the temperature of the vitrification mixture will fall below the T_{g} of the vitrification medium to allow the vitrification of the biological sample.

Maintaining the low atmospheric pressure can require containing the vitrification mixture in a sealed enclosure, such as a desiccation chamber. It will be appreciated by those skilled in the art that providing and/or maintaining a low atmospheric pressure around the vitrification mixture will typically require that the desiccation chamber be capable of withstanding the low pressure therein. Such can be of any suitable or desired shape and/or material, being constrained by a requirement to maintain a low atmospheric pressure therein, requiring a sufficient seal and sufficient wall strength. The desiccation chamber can be operably connected to a vacuum source to lower the atmospheric pressure therein, while further allowing air to return upon vitrification completion. The desiccation chamber may be sufficiently sealed or closed so as to allow for an applied vacuum to effectively lower the atmospheric pressure in the desiccation chamber to the desired range.

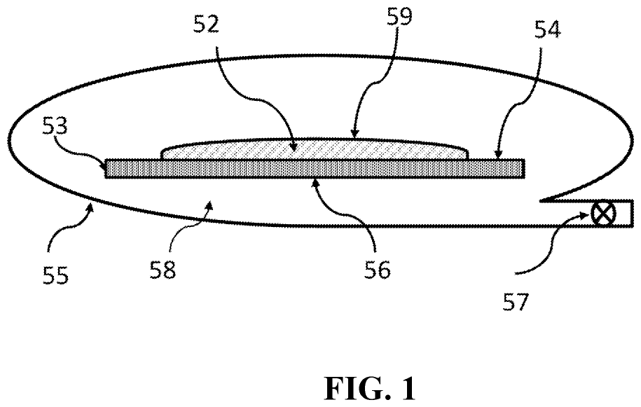

In some aspects, the vitrification mixture is placed on or in a capillary network to enhance vitrification of the vitrification mixture. A capillary network can prevent the vitrification mixture from boiling under a reduced atmospheric pressure. The principles of capillary assisted evaporation are described in U.S. Pat. No. 10,568,318. Briefly, FIG. 1 illustrates favorable conditions for capillary assisted drying of a vitrification mixture that includes: a plurality of capillary channels forming a capillary plate/membrane 53, the capillary channels having a first opening 54 and a second opening 56; placing a vitrification mixture 52 on the first opening 54, further exposing the second openings 56 and the vitrification mixture's surface 59 to a surrounding atmosphere 58 having lower humidity than the vitrification mixture; optionally applying heat to the vitrification mixture and desiccating away the said vitrification mixture by capillary action until the vitrification mixture enters into a glassy state thereby preserving the bioactive agent in the glassy state. The chemistry, humidity, pressure and temperature inside the enclosure 55 is controlled through a control mechanism 57. In some aspects, the amount of vitrification mixture is sufficiently low such that the entire vitrification mixture may be held in the pores of the capillary network thereby improving overall desiccation rates and preservation of the bioactive agent.

The control mechanism 57 is simplified for illustration purposes only and can have multiple systems and mechanisms to attain the most favorable conditions for desiccation and vitrification. Optionally, a second capillary plate/membrane similar to 53 is placed directly on top of the vitrification mixture 52 to benefit from the capillary assisted drying method of the present disclosure at the top surface of vitrification mixture 52. However, gravity will not favor the capillary force on the top. In some aspects, a flow of low humidity gas (less than 30% relative humidity) is provided across the second openings 56 of the capillary plate/membrane so as to enhance the capillary effect. Inert or relatively inert gases such as nitrogen, argon, xenon, or others may be used as a low humidity gas. In some aspects, a reduced pressure or vacuum is maintained inside the enclosure 55. In some aspects, a suction force/pressure is provided across the second opening 56 to achieve increased desiccation speed. It is to be noted that, maintaining a low humidity surrounding (optionally 5% relative humidity or less) is essential to prevent rehydration after desiccation has been performed.

In some aspects, a capillary network is provided by an undulating or irregular surface structure of a membrane or surface whereby the troughs in the undulating surface itself may function as a capillary network for the vitrification of a bioactive agent. The capillary network may be of sufficient thickness to restrict liquid or fluid from accumulating on the surface thereof. To realize the capillary effect the liquid may be accommodated within the pores of the membrane forming a meniscus. The liquid fraction (ξ) at the capillary interface, i.e., the volume occupied by the liquid is a parameter for consideration to promote improved capillary evaporation. Capillary driven evaporation occurs when the viscous pressure drop in the liquid surpasses the maximum capillary pressure at the liquid-vapor interface. The liquid fraction is related to the overall pressure drop from the bulk to the liquid-vapor interface. Under atmospheric pressure and no applied heat flux, the liquid covers large fraction, leading to a liquid fraction, ξ→1. Under these conditions, the capillary driven evaporation rate is minimal. Reducing the ambient pressure as shown in reduces and in turn increases the evaporation rate. However, beyond certain threshold pressure drop, nucleation boiling can occur which is undesirable. An applied heat flux Q can also enhance the evaporation rate, but the risk of film boiling exists, which is also undesirable. Applying the heat flux from the surface of the capillary meniscus, eliminates or reduces the risk of film boiling. Under large ΔP and Q applied in a counter gradient fashion, the liquid meniscus is confined to the pores, i.e., the liquid fraction ξ<<1 (˜0.25), resulting in highest evaporation rate while avoiding boiling (ξ=πd²/4p², where p is distance between ridges or height of the membrane and d the diameter of the circle formed by the shape of the liquid meniscus). Therefore, maintaining a temperature gradient between the surface and the bulk liquid leads to capillary evaporation, where the fast evaporation can be achieved. As the liquid level recedes into the capillary membrane, capillary evaporation phenomena is still realized as long as the pressure gradient and temperature gradients are maintained. In some aspects, a capillary network under the vitrification mixture may assist in the evaporative processes during desiccation.

In some aspects, the heat energy is applied to a vitrification mixture as it undergoes vitrification on a capillary network. An underlying capillary network can allow for even and complete vitrification of a vitrification mixture receiving heat energy while protecting the vitrification mixture from boiling. The capillary network can be a continuous network of capillaries. In some instances, the capillary network can be continuous capillary channels forming an uninterrupted structure from a first end to a second end. In some instances, the capillary network can be provided by an underlying porous material, such as a membrane, or an underlying contoured or ridged surface wherein the troughs and peaks thereof provide a bed sufficient to subject a liquid vitrification mixture to capillary action during vitrification.

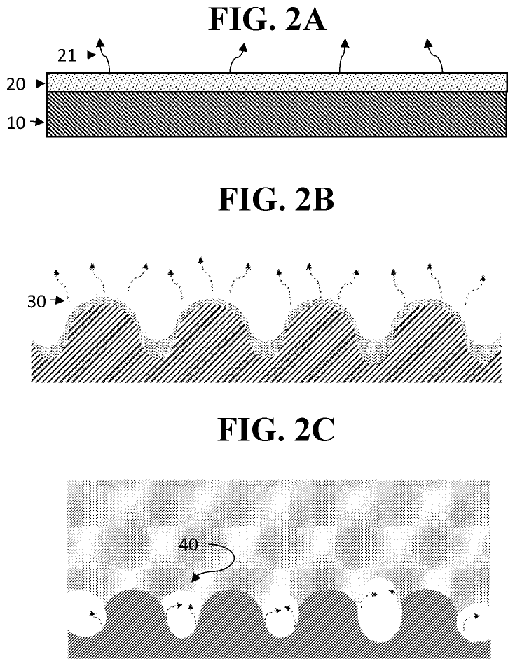

With reference to FIG. 2A, depicted is a continuous hydrophilic bed 10 covered by application of a thin liquid layer of vitrification mixture 20. In some aspects, a protective fluid layer of vitrification mixture may protect the biological sample from boiling during exposure to low atmospheric pressure. Prevention of boiling under reduced atmosphere can be avoided and/or reduced with an extremely thin liquid film on a hydrophilic surface as shown in FIG. 2A. However, while prevention of boiling is possible, due to the limitation in thickness of the liquid the available surface area reduces the amount of liquid that can be vitrified. The presence of a contoured surface, such as that set forth in FIG. 2B, effectively provides a surface upon which the vitrification mixture can be subjected to a capillary action due to preferential desiccation occurring at the peaks thereby drawing moisture up during the vitrification process and that can similarly protect the biological sample (that sits predominantly on the apices of the contours or ridges) from boiling and allows for significantly large sample volumes to be vitrified. Further, as the sample vitrifies at the peaks of the contours, capillary action draws fluid from the underlying trough, thereby promoting excellent vitrification and desiccation of the vitrification mixture. Similarly, if a porous material of a membrane of capillaries supports the biological sample within the capillaries, capillary action will draw fluid from the capillary channels during the vitrification process and provide even and complete vitrification and desiccation of larger volumes of biological sample. However, as set forth in FIG. 2C, if the capillary action cannot successful draw fluid up, such as in the case of a fluid loading that is too great, the liquid fills the surface patterns or is retained in the troughs, where bubble nucleation and boiling becomes dominant under reduced pressure which may lead to damage of sensitive molecules contained therein.

Accordingly, in some aspects of the present disclosure, the volume of fluid present in the vitrification mixture can be established such the fluid can fill the capillary network without overflowing or pooling on the surface.

In certain aspects, the desiccation chamber or membrane contained therein may be suitably patterned such that the walls of the chamber or membrane provide a capillary network to the vitrification mixture when placed therein. For example, a contour or ridge such as that depicted in FIG. 2B can line the walls of a chamber to provide an underlying capillary network. In other aspects, a porous material of a continuous capillary network with a vitrification mixture therein is provided into a sealable desiccation chamber. In certain aspects, a porous material may include a membrane of a plurality of continuous capillary channels.

A capillary membrane may be placed in a support that may itself be heated to avoid the vitrification mixture from experiencing a temperature below the Tf. In some aspects, a support scaffold may be included between the membrane and the heating block or side of any chamber in which the membrane is located to separate the membrane from the surface from which heat is produced. This prevents direct heating from beneath the sample to substantially allow for desiccation from two directions or avoiding the greater heat exposed to the troughs of the membrane material, in some aspects.

A heating element may be instead or in addition introduced within the chamber allowing for directed heat application to the surface of the vitrification membrane thereby promoting effective vitrification at the desired locations within/on the membrane to promote excellent capillary action and prevent boiling of the vitrification medium during vitrification.

Optionally, a supporting substrate may be utilized to provide and/or transfer heat energy to the vitrification mixture. It will be appreciated by those skilled in the art that to provide heat energy effectively to the vitrification mixture, the supporting substrate may in some aspects be of a good conducting material, such as a metal. In other aspects, a porous material to provide a continuous network of capillaries may be located between the vitrification mixture and the underlying solid support substrate.

As illustrated in FIG. 2, the presence of contours and/or ridges provides capillary ridges to enhance vitrification. The presence of a vitrification mixture over the surface allows for fast evaporation from the peaks and by drawing the vitrification mixture toward the peaks by capillary action. The presence of a continuous capillary network further allows the fluid volume of the vitrification mixture to evenly evaporate and prevent boiling while also preventing excess fluid build-up, which can also experience damaging boiling. Similarly, a porous material such as a membrane of continuous capillary channels may provide an underlying capillary network. In such aspects, a porous material, such as a membrane, is housing the vitrification mixture and the capillary action therein provides for enhanced vitrification. Accordingly, in some aspects of the present disclosure, the vitrification mixture is placed on a continuous capillary network. In further aspects, the vitrification mixture is placed on a patterned and/or ridged and/or contoured optionally porous material. In further aspects, the continuous capillary network is formed by patterns and/or ridges and/or contours within walls of the desiccation chamber. In other aspects, the capillary network is provided by a porous material including a plurality of continuous capillary channels.

The desiccation chamber should further be capable of or arranged to house the vitrification mixture therein. In some aspects, the desiccation chamber should be capable of housing the vitrification mixture on a porous material and/or a supporting substrate. In some aspects, the vitrification mixture is prepared for vitrification by placement upon a substrate. In some aspects, the substrate may be a porous material, such as a membrane and/or a bed of arranged capillaries. In further aspects, the walls of the desiccation chamber serve as a supporting substrate and are ridged and/or patterned and/or contoured to provide a capillary network therein.

Capillaries in a capillary network membrane can provide an interface for rapid evaporation. The capillary network formed from either an underlying patterned ridged support or of a porous material such as a membrane may be made of a material that is not toxic and not reactive to the biomaterials or biological samples and does not react chemically or physically with the vitrification medium. The material can be of a suitable polymer, metal, ceramic, glass, or a combination thereof. In some aspects, a continuous capillary network is formed from a material of polydimethylsiloxane (PDMS), polycarbonate, polyurethane, polyethersulphone (PES), polyester (e.g. polyethylene terephthalate), among others. Illustrative examples of a capillary channel containing membrane suitable as a surface in the devices and processes provided herein include hydrophilic filtration membranes such as those sold by EMD Millipore, Billerica, MA. In certain aspects, the porous material does not substantially bind, alter, or otherwise produce a chemical or physical association with a component of a biological sample and/or vitrification medium. The porous material is optionally not derivitized. Optionally, capillary channels may be formed in a substrate (e.g. desiccation chamber walls) of desired material and thickness by PDMS formation techniques, laser drilling, or other bore forming technique as is known in the art.

A capillary network under the vitrification mixture or housing the vitrification mixture may assist in the evaporative processes during desiccation. As described herein, capillaries may be provided by patterning or contouring the walls of a desiccation chamber to effectively provide an underlying capillary bed or by providing a porous material of a continuous capillary network, such as with a membrane. In some aspects, the capillary network provided by a porous material and/or a patterned and/or contoured surface features pores of about 20 μm or less, such that the pores provide underlying capillaries to assist in vitrification. In some aspects, the pores may be of an average opening of from about 20 μm to about 0.1 μm, including about 19, 18, 17, 16, 15, 14, 13, 12, 11, 10, 9, 8, 7, 6, 5, 4, 3, 2, 1.0, 0.9, 0.8, 0.7, 0.6, 0.5, 0.4, 0.3, and 0.2 μm. A capillary channel may have a length optionally defined by the thickness of a substrate that forms the channels or by one or a plurality of individual channels themselves. A capillary channel length is optionally about one millimeter or less, but is not to be interpreted as limited to such dimensions. Optionally, a capillary channel length is of about 0.1 microns to about 1000 microns, or any value or range therebetween. Optionally, a capillary channel length is of about 5 to about 100 microns, optionally of about 1 to about 200 microns, and/or optionally of about 1 to about 100 microns. A capillary channel length is optionally about 5, 10, 15, 20, 25, 30, 35, 40, 45, 50, 55, 60, 65, 70, 75, 80, 85, 90, 95, or 100 microns. In some aspects, the length of the capillary channels varies throughout a plurality of capillary channels, optionally in a non-uniform variation.

The cross-sectional area of the capillary channel(s) may be of about 2000 μm² or less. Optionally a cross-sectional area is of about 0.01 μm² to about 2000 μm², optionally of about 100 μm² to about 2000 μm², or any value or range therebetween. Optionally, a cross-sectional area of the capillary channel(s) is of about 100, 200, 300, 400, 500, 600, 700, 800, 900, 1000, 1100, 1200, 1300, 1400, 1500, 1600, 1700, 1800, 1900, or 2000 μm² or less.

Capillary assisted evaporation rate may be affected by both atmospheric demand (humidity, temperature and velocity of air/gas at the evaporating surface), and (i) the characteristics of the capillary channels that generate the driving capillary force, (ii) the liquid meniscus depth, and (iii) the viscous resistance to flow through the capillary. Consequently, complex and highly dynamic interactions between capillary properties, transport processes, and boundary conditions result in wide range of evaporation behaviors. For fast drying the key parameters may include: (1) the conditions that support formation and sustain a liquid network at the evaporating surface and (2) the characteristics that promote formation of capillary pressure that induce sufficient flow to supply water at the evaporating surface.

In some aspects, the porous material may be ridged and/or contoured or placed upon a ridged and/or contoured underlying support substrate, such that the porous material adopts a similar shape when placed or pressed thereon. As depicted in FIG. 2B, the contours and/or ridges of a patterned material may increase surface area to provide for increased exposure for evaporation.

In some aspects, the capillary network is of a hydrophilic material. In other aspects, the capillary network may be of a hydrophobic material and further treated to be hydrophilic or more hydrophilic in nature, such as through exposure to a plasma.

In some aspects, the bioactive agent is placed, covered, or mixed in a vitrification medium to form a vitrification mixture. The presence of appropriate vitrification agents in a vitrification medium can be essential as the vitrification medium desiccates under the surrounding conditions as set forth herein. Fast desiccation methods by themselves do not necessarily assure success in the viability of the bioactive agent following desiccation. A vitrification medium that forms glass and/or that suppresses the formation of crystals in other materials may be required. A vitrification medium may also provide osmotic protection or otherwise enable cell survival during dehydration of the biological sample. Illustrative examples of agents to include in a vitrification medium may include one or more of the following: dimethylsulfoxide, glycerol, sugars, polyalcohols, methylamines, betines, antifreeze proteins, synthetic anti-nucleating agents, polyvinyl alcohol, cyclohexanetriols, cyclohexanediols, inorganic salts, organic salts, ionic liquids, or combinations thereof. In some aspects, a vitrification medium optionally contains 1, 2, 3, 4, or more vitrification agents.

A vitrification medium may include a vitrification agent at a concentration that is dependent on the identity of the vitrification agent. Optionally, the concentration of the vitrification agent is at a concentration that is below that which will be toxic to the biological sample being vitrified where toxic is such that functional or biological viability is not achieved upon subsequent sample use. The concentration of a vitrification agent is optionally of about 500 μM to about 6 M, or any value or range therebetween, including about 1, 2, 3, 4, or 5 M. For the vitrification agent trehalose, the concentration is optionally of about 1 M to about 6 M, including 2, 3, 4, or 5 M. Optionally, the total concentration of all vitrification agents when combined is optionally of about 1M to about 6M, including 2, 3, 4, or 5 M

Trehalose, a glass forming sugar, has been employed in anhydrous vitrification and may provide desiccation tolerance in several ways. However, vitrified 1.8 moles/liter (M) trehalose in water has a glass transition temperature of −15 to 43° C. To achieve vitrification above 0° C., higher concentrations (6-8 M) are required which could be damaging to the biological materials. Alternatively, the vitrification medium may include buffering agents and/or salts to increase the Tg value of the VM. In some aspects, a vitrification medium may optionally include water or a solvent and/or a buffering agent and/or one or more salts and/or other components. A buffering agent may be any agent with a pKa of about 6 to about 8.5 at 25° C. Illustrative examples of buffering agents may include HEPES, TRIS, PIPES, MOPS, among others. A buffering agent may be provided at a concentration suitable to stabilize the pH of the vitrification medium to a desired level.

A vitrified medium including 1.8 M trehalose, 20 mM HEPES, 120 mM ChCl, and 60 mM betine provides a glass transition temperature of +9° C. An exemplary vitrification medium for the capillary assisted vitrification may include trehalose, and one or more buffering agents containing large organic ions (>120 kDa) such as choline or betine or HEPES as well as buffering agent(s) containing small ions such as K or Na or Cl.

The bioactive agent may be coated or immersed in a vitrification medium and placed on a support substrate to retain the vitrification medium during the steps of vitrification as set forth herein. In certain aspects, the capillary network absorbs some of the vitrification mixture while allowing a thin layer of fluid to remain on the surface.

In some instances, the biological sample may be coated and/or mixed with a vitrification medium in the desiccation chamber. In other aspects, the biological sample may be prepared with a vitrification medium prior to placement within the desiccation chamber.

In some aspects, the methods of the present disclosure may be performed for a desiccation time. A desiccation time is a time sufficient to promote suitable drying to vitrify the vitrification medium. A desiccation time is optionally from about 1 second to about 1 hour, including but not exceeding about 10 s, 30 s, 1 min, 5 min, 10 min, 20 min, 25 min, 30 min, 35 min, 40 min, 45 min, 50 min and 55 min. Optionally, a desiccation time is of from about 1 second to about 30 min, optionally of from about 5 seconds to about 10 min.

A bioactive agent may remain viable in vitrified state during storage above cryogenic temperature may vary in time from one sample material to the next. Optionally, a bioactive agent may remain viable while in storage above cryogenic temperature for 2-20 days. In other aspects, a bioactive agent may remain viable while in storage above cryogenic temperature for 10 weeks. In other aspects, a bioactive agent may remain viable in storage above cryogenic temperature for up to one year. In other aspects, a bioactive agent may remain viable while in storage above cryogenic temperature for up to 10 years.

Alternatively, after vitrification above cryogenic temperatures employing the teachings and devices of the current disclosure, the vitrified bioactive agent can be stored at cryogenic temperatures and/or in liquid nitrogen for very long periods. For many bioactive agents, this is a preferred approach to avoid cryoinjury that commonly occurs during direct vitrification at cryogenic temperatures. A preferred approach in one aspect is to vitrify the bioactive agent at room temperatures utilizing low concentrations of vitrification agents (e.g. <2 M trehalose) and then immediately store at cryogenic temperatures. Therefore, the said device is optionally made out of materials storable at a temperature between −196° C. to 60° C. following the vitrification according to the teachings of the current disclosure.

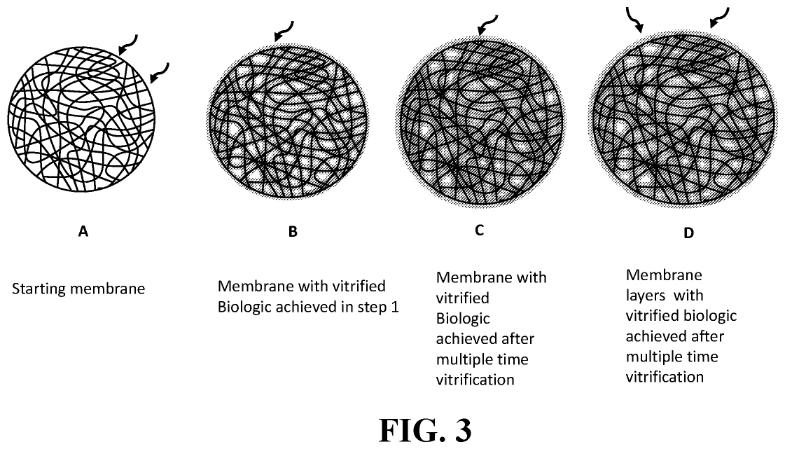

Optionally, a bioactive agent is vitrified on or in the vitrification membrane in sequential layers. It was found that not only does serial vitrification boost the amount of bioactive agent that may be stored on in the membrane prior to reconstitution, but also serial vitrification promotes successful concentration of viable and active bioactive agent following reconstitution in an administration solvent. As illustrated in FIG. 3, vitrification of a bioactive agent in the channels of a membrane may be achieved by adding a vitrification mixture that includes the bioactive agent and a vitrification medium into the channels of the membrane and subjecting the membrane to desiccation as provided herein until glass formation occurs on the walls of the channels. A second vitrification mixture may then be added to the same membrane, optionally to a level what fills or the remaining channels or less, and a second desiccation process is performed to add additional bioactive agent, or a different bioactive agent, cofactor, coenzyme, or other desired molecule(s), to the channels in vitrified state. The addition of more vitrification mixture with more of the same or different bioactive agent may be added 1, 2, 3, 4, 5, 6, or more times. In some aspects, the number of times additional vitrification medium can be added is limited by the requirement that the membrane with vitrified material therein still has sufficient pore structure or shaping to allow absorption of additional vitrification mixture into or onto the channels of the membrane.

A formulation as provided herein is formed by reconstituting the vitrified bioactive agent in an administration solvent at such a volume that the rehydration of the bioactive agent creates a fluid that creates a supersaturated fluid of the bioactive agent in the administration solvent. The amount of administration solvent is optionally less than the amount of vitrification medium used to prepare the vitrified sample such that the reconstituted bioactive agent is present in the administration solvent at a higher concentration than in the vitrification mixture.

The amount of administration solvent used may be tailored to the number of doses of bioactive agent are desired. For example, for subcutaneous dosing, an administration solvent may be added to a volume of 5 ml or less, optionally 4 ml or less, optionally 3 ml or less. In some aspects, a formulation is administered at a volume that is tolerated without pain in a human subject, optionally 2.5 ml or less, optionally 2.0 ml or less, optionally 1.5 ml or less. In other aspects, an administration solvent is added at a volume in excess of 5 ml depending on the desired downstream dosing such as for intravenous or intramuscular injection.

An administration solvent is termed an administration solvent for exemplary purposes alone. The composition of the solvent need not be, but may be, formed of only acceptable materials for administration to a subject, but may include other materials in addition thereto or substitution thereof depending on the intended downstream use of the bioactive agent. An administration solvent may be as described herein.

### Methods of Prevention or Treatment of a Disease or Condition

A formulation as provided herein may be used to prevent or treat a disease or condition or a symptom of a disease or condition. The identity of the disease or condition that may be prevented, treated, or other will depend on the type of biological activity that is performed by the bioactive agent upon administration to a subject that has the disease or condition or is at risk for contracting or exhibiting a disease or condition.

Optionally, a disease or condition for which the formulation as provided herein may be administered includes but is not limited to: rheumatoid arthritis; cancers such as but not limited to breast cancer, lung cancer, colorectal cancer, pancreatic cancer, leukemia, among others; autoimmune diseases; acute organ rejection; ankylosing spondylitis; inflammatory diseases; hypercholesterolemia; Crohn's disease; ulcerative colitis; psoriasis; diabetes; multiple sclerosis; heart attack; systemic lupus erythematosus; age related macular degeneration; diabetic retinopathy, pneumonia; anemia; chronic migraine; infectious diseases; hepatitis B, hemophilia, respiratory syncytial virus infection; HPV infection; varicella virus infection; growth hormone deficiency; osteoporosis; asthma; allergic asthma; chronic idiopathic urticarial; infertility; HIV infection; meningococcal disease; cystic fibrosis; paroxysmal nocturnal hemoglobinuria; a procoagulant condition; myocardial ischemia; myocardial infarction; Alzheimer's disease; among others.

A formulation as provided herein is optionally administered by any desired route suitable for administration of the bioactive agent. Administration is optionally subcutaneously, intravenously, intramuscularly, or other. In particular aspects, the formulation is administered to a subject subcutaneously. The increased concentration of the bioactive agent in the administration solvent allows for lower volume dosing while providing sufficient and proper dosing amounts of the bioactive agent. When dosing subcutaneously, the formulation is optionally administered without the presence or requirement for coadministration of hyaluronidase. In some aspects, however, hyaluronidase is present when the bioactive agent is administered.

The formulation is optionally administered once or more daily, weekly, monthly, or annually. Optionally, the formulation is administered once daily. The frequency of administration is as is directed for the bioactive agent of interest.

The bioactive agent is optionally an antibody at a concentration of 50 mg/ml or greater in the administration solvent and the formulation is administered subcutaneously. Optionally, the antibody at a concentration of 90 mg/ml or greater in the administration solvent and the formulation is administered subcutaneously. Optionally, the antibody at a concentration of 150 mg/ml or greater in the administration solvent and the formulation is administered subcutaneously. The administration solvent is optionally buffered saline or normal saline.

The bioactive agent is optionally an anti-proprotein convertase subtilisin/kexin type 9 (PCSK9) antibody at a concentration of 50 mg/ml or greater in the administration solvent and the formulation is administered subcutaneously. Optionally, the antibody at a concentration of 90 mg/ml or greater in the administration solvent and the formulation is administered subcutaneously. Optionally, the antibody at a concentration of 150 mg/ml or greater in the administration solvent and the formulation is administered subcutaneously. The administration solvent is optionally buffered saline or normal saline.

The bioactive agent is optionally an antibody-drug (toxin) conjugate at a concentration of 50 mg/ml or greater in the administration solvent and the formulation is administered subcutaneously. Optionally, the antibody at a concentration of 90 mg/ml or greater in the administration solvent and the formulation is administered subcutaneously. Optionally, the antibody at a concentration of 150 mg/ml or greater in the administration solvent and the formulation is administered subcutaneously. The administration solvent is optionally buffered saline or normal saline.

Various aspects of the present disclosure are illustrated by the following non-limiting examples. The examples are for illustrative purposes and are not a limitation on any practice of the present invention. It will be understood that variations and modifications can be made without departing from the spirit and scope of the invention.

### EXAMPLES

### Example 1

Polyethylene terephthalate membranes were prepared for vitrification of an exemplary antibody IgG by treatment in 0.05 wt % phosphate buffered saline with Tween 20 by immersion and continuous stirring at 25° C. for 20 minutes. The membranes were then dried in an oven at 55° C. for one hour.

A vitrification mixture was prepared by combining 20 mg/mL Powdered IgG (IHUIGGGGLYIGM-34158, Innovative Research, Inc.), 5 wt % Trehalose dihydrate, 0.25 wt % Glycerol, in phosphate buffered saline. 150 microliters of the vitrification mixture was added to the treated membranes. The membranes were placed on a metal screen within a vitrification chamber and desiccated for 30 minutes on a bed temperature of 37° C. The chamber in each case was evacuated to 29.5 mmHg. An additional 150 microliters of vitrification medium was then added to the membrane and the vitrification process repeated. This was further repeated for a total of 5 vitrification cycles. A total of three separate samples were created by the above process.

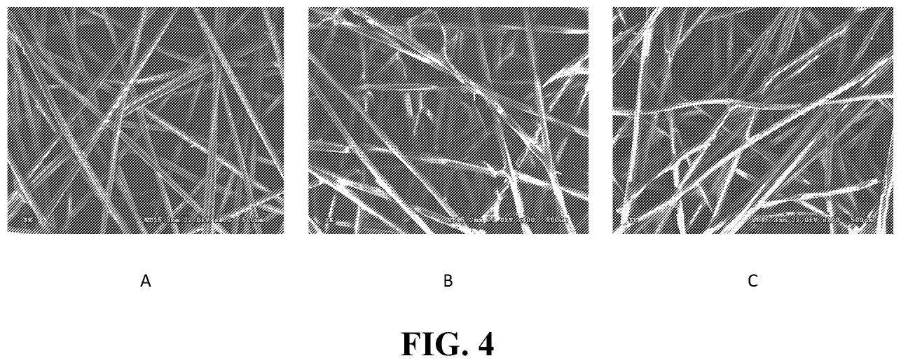

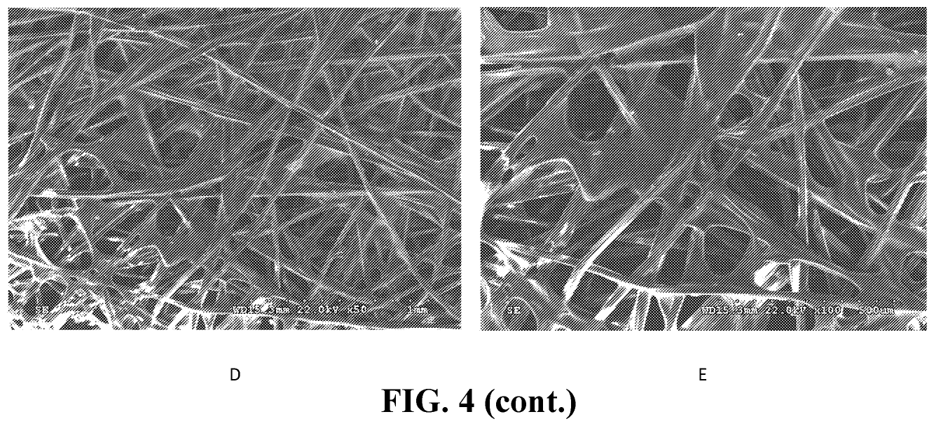

The resulting membranes were subjected to scanning electron microscopy (SEM) to analyze the collection of vitrified antibody in the channels of the membrane. Results are illustrated in FIG. 4A illustrates the structure of the membrane prior to vitrification. FIG. 4B illustrates the membrane following the first round of vitrification of the antibody and showing initial deposits/coating of antibody on the fibers of the membrane. FIG. 4C illustrates the membrane following the second round of vitrification illustrating the further collection of vitrified material. FIG. 4D illustrates the membrane following the third round of vitrification which is shown at 100× greater magnification in FIG. 4E both illustrating further filling of the pores and greater continuity of the coating of vitrified antibody within the channels of the membrane.

The vitrified antibody containing membranes were then contacted with 150 microliters of water and incubated for 5 minutes. The membranes were then rolled into a tube shape with the mesh support and centrifuged at 10×g for 10 minutes to collect all the reconstituted antibody.

The reconstituted antibody was then assayed for concentration by BCA protein assay by standard techniques. The starting concentration of antibody in the vitrification mixture was 20 mg/ml. Following vitrification and reconstitution as described, the antibody concentration was 91.59 mg/ml with a standard error of 1.82 mg/ml. This reveals a greater than 4.5-fold increase in concentration of the antibody and demonstrates the efficacy of superconcentrating antibody above and beyond that which is typically available for intravenous delivery antibody biologics.

### Example 2

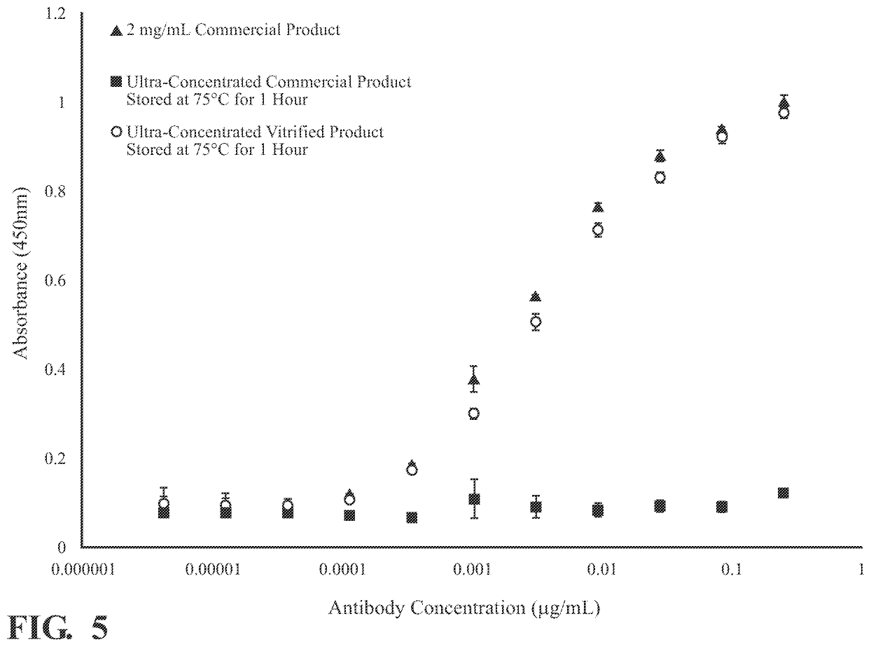

The vitrified materials of Example 1 were analyzed for functional stability of the antibody. The vitrified samples of Example 1 were subjected to heat treatment at 75° C. for one hour. This sample was then concentrated as per Example 1 and subjected to analysis by ELISA. Briefly, polystyrene plates were coated with anti-IgG antibody. The reconstituted sample was serially diluted and incubated with the coated plates. After washing, detection of the presence or absence of the reconstituted antibody were performed by a second anti-IgG antibody bound to horseradish peroxidase. Antibody sandwich detection was performed using a TMB substrate. As a control, identical antibody solution not subjected to vitrification was generated to 2 mg/ml and diluted to the identical concentrations and subjected to the same ELISA analysis. As a second control, antibody solution at 2 mg/ml was heat treated to 75° C. for one hour and subjected to the same ELISA analysis. As is illustrated in FIG. 5, the vitrified antibody reconstituted to 91.59 mg/ml maintained structure and solvation identical to unvitrified to the not heat treated control as illustrated by substantially overlapping detection levels at each identical concentration. In contrast, the heat treated solution sample that was not vitrified was substantially destroyed by the heat treatment.

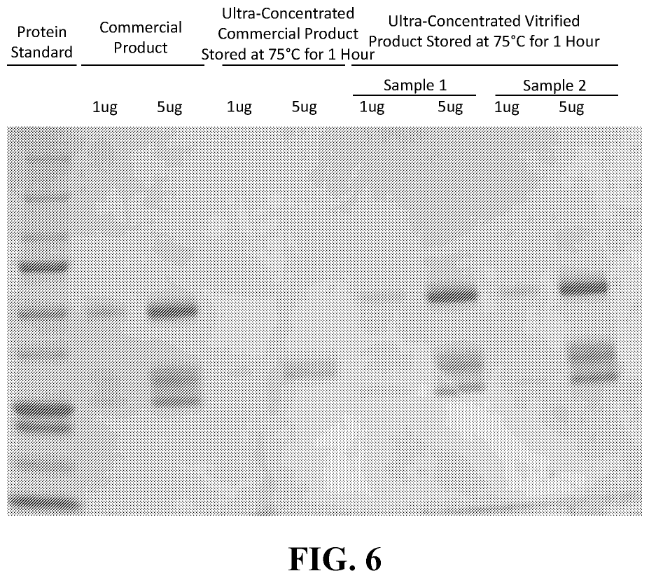

In a separate set of studies, the test sample, as received 2 mg/ml solution and heat treated solution were subjected to SDS-PAGE and detected using Coomassie brilliant blue. Lanes were loaded with either 1 μg or 5 μg of sample antibody. As is illustrated in FIG. 6, the ultraconcentrated vitrified heat treated sample maintains intact heavy and light chain configurations whereas the unvitrified and heat-treated antibody is nearly totally degraded. In sum, these studies reveal that vitrification maintains the functional conformation of the antibody despite the creation of the superconcentrated reconstituted solution of antibody, as well as the clear heat stability of the antibody when in the vitrified state when prepared as provided herein.

### Example 3

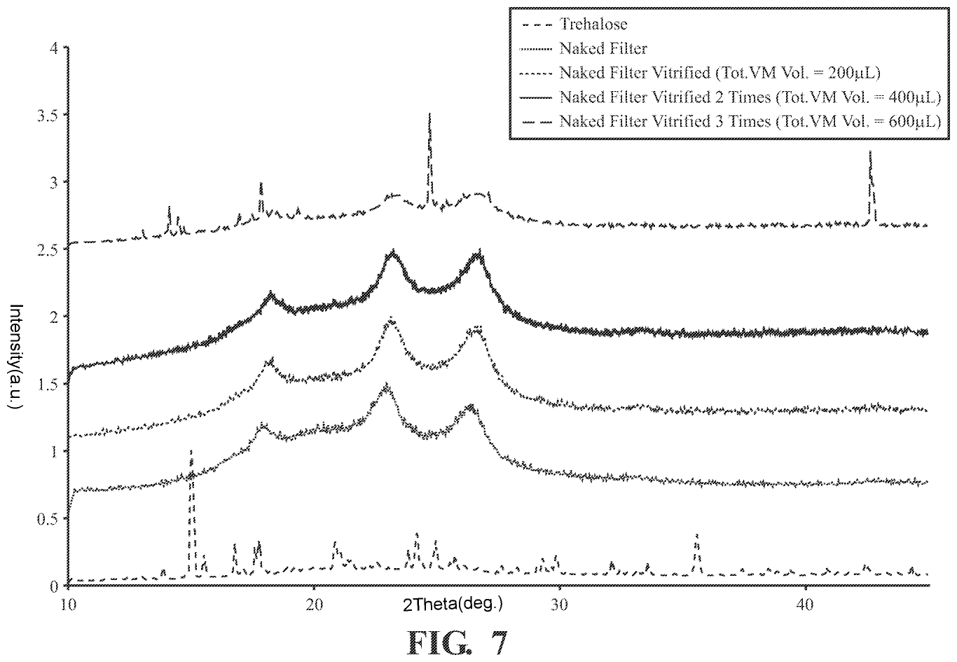

The antibody of Example 1 was vitrified under the Example 1 conditions with the exception of the use of 200 μl of sample was added to each membrane. Following each serial vitrification, the membrane with the vitrified material therein was subjected to analysis by x-ray diffraction (XRD). As is illustrated in FIG. 7, dried trehalose alone showed a representative crystalline structure. The filter membrane itself, prior to vitrification of any material, showed a characteristic amorphous structure as expected. Following the first vitrification, which contained vitrified material as is illustrated in FIG. 4B, maintained this amorphous structure indicating the presence of well vitrified material absent crystalline structure formation during vitrification. Similar amorphous structure was observed following the second round of vitrification onto the same membrane. In contrast, the third round of vitrification showed the presence of some level of crystalline structure formation indicating that the amount of solution was too great to be housed in the channels of the membrane due to prior vitrified material occupying the space. Under the third round conditions, crystal formation will occur during the desiccation of the surface lying material.

These data indicate that the number of serial vitrification rounds and overall amount of material that may be housed in the membrane and still maintain effectively stored vitrified biological agent is limited by the presence of adequate pore volume to house the vitrification mixture during vitrification and that prior to overloading the membrane, amorphous structure is maintained indicating the presence of adequately stabilized biological agent.

The following listing of exemplary aspects also relate to the present disclosure.

Aspect 1. A superconcentrated formulation of a bioactive agent comprising: a bioactive agent and an administration solvent, said bioactive agent at a concentration higher than the concentration of said bioactive agent in said administration solvent formulated for subcutaneous administration to a subject, optionally at a concentration higher than a concentration at which physiologically relevant aggregation of the bioactive material in the administration solvent would occur absent preparation by a process as provided herein, optionally by a preparation process of any one of aspects 14-27, or wherein the concentration of the bioactive agent in the administration solvent has similar or identical colloidal stability, liquid-liquid phase separation or propensity for gel formation relative to an aggregate free concentration of said bioactive agent in said administration solvent absent preparation as provided herein or as by any one of aspects 14-27.

Aspect 2. The superconcentrated formulation of aspect 1, wherein said solvent is aqueous.

Aspect 3. The superconcentrated formulation of any of aspects 1 and 2, wherein said solvent comprises water and one or more monovalent or divalent salts.

Aspect 4. The superconcentrated formulation of any of aspects 1-3, wherein said bioactive agent is present at a concentration above a concentration that said bioactive agent agglomerates in said solvent.

Aspect 5. The superconcentrated formulation of any one of aspects 1-4, wherein said bioactive agent is present at a concentration in excess of 50 mg/mL, optionally in excess of 90 mg/mL, optionally in excess of 100 mg/mL, optionally in excess of 150 mg/mL.

Aspect 6. The superconcentrated formulation of any one of aspects 1-5 further comprising one or more pharmacologically acceptable excipients.

Aspect 7. The superconcentrated formulation of any one of aspects 1-6, wherein said formulation is in a volume up to 2.5 milliliters.

Aspect 8. The superconcentrated formulation of any of aspects 1-7, excluding a hyaluronidase

Aspect 9. The superconcentrated formulation of any of aspects 1-8, wherein said bioactive agent is an antibody, protein, lipid particle, optionally a lipid nanoparticle, or other therapeutic agent.

Aspect 10. The superconcentrated formulation of aspects 1-9 wherein said bioactive agent is an antibody, optionally a humanized antibody.

Aspect 11. The superconcentrated formulation of aspect 9 or 10, wherein said antibody comprises muromonab-CD3, infliximab, rituximab, solanezumab, bapineuzumab, catumaxomab, trastuzumab, cetuximab, omalizumab, adalimumab, bevacizumab, BAN2401, tositumomab, or alemtuzumab.

Aspect 12. The superconcentrated formulation of any one of aspects 1-9, wherein said bioactive agent is a protein, wherein said protein comprises an interleukin or an interferon, optionally interferon β-1b, Peginterferon alfa-2b, Roferon-A, or aldesleukin.

Aspect 13. The superconcentrated formulation of any one of aspects 1-12, wherein said formulation has a viscosity of 20 cP or less, optionally 12 cP or less, optionally 8 cP or less, optionally 2 cP, or less, optionally 1 cP or less, wherein said viscosity is measured at 20° C. and one atm.

Aspect 14. A process of preparing the superconcentrated formulation of any one of aspects 1-13, comprising: a) overlaying a vitrification mixture comprising a bioactive agent and a vitrification medium on a membrane comprising a capillary network, said membrane in a desiccation chamber; b) lowering the atmospheric pressure within the desiccation chamber; c) providing a heat energy to the vitrification mixture, wherein the heat energy is sufficient to prevent the vitrification mixture from entering a cryogenic state; d) desiccating the vitrification mixture by capillary action until the vitrification mixture enters a glassy state; e) reconstituting said bioactive agent in the administration solvent, whereby the concentration of said bioactive agent in said administration solvent is greater than or equal to a concentration of said bioactive agent in said administration solvent when formulated for subcutaneous administration to a subject, optionally greater than or equal to a concentration of said bioactive agent in said vitrification mixture, optionally greater than a concentration of said bioactive agent in said vitrification mixture.

Aspect 15. The process of aspect 14, further comprising repeating steps a-d prior to step e.

Aspect 16. The process of aspect 15, wherein said repeating is 1-3 times.

Aspect 17. The process of any one of aspects 14-16, wherein the capillary network is provided by contours along the surface of the membrane.

Aspect 18. The process of any one of aspects 14-17, wherein the capillary network within the desiccation chamber is supported by an underlying solid support substrate.

Aspect 19. The process of any one of aspects 14-18, wherein vitrification of the vitrification mixture occurs in less than 30 minutes, optionally less than 10 minutes.

Aspect 20. The process of any one of aspects 14-19, wherein the heat energy is provided by heating the vitrification mixture.

Aspect 21. The process of any one of aspects 14-20, wherein the atmospheric pressure is lowered to a value of from about 0.9 atm to about 0.005 atm.

Aspect 22. The process of aspect 21, wherein the atmospheric pressure is lowered to about 0.004 atm.

Aspect 23. The process of any one of aspects 14-22, wherein the heat energy provided is sufficient to prevent crystallization of the bioactive agent within the vitrification mixture during vitrification.

Aspect 24. The process of any one of aspects 14-23 wherein the provided heat energy is sufficient to keep the bioactive agent at a temperature of from about 0° C. to about 40° C. during said vitrifying.

Aspect 25. The process of any one of aspects 14-24, wherein said vitrification medium comprises trehalose, glycerol and betine and/or choline.

Aspect 26. The process of any one of aspects 14-25, wherein the capillary network is hydrophilic.

Aspect 27. The process of any one of aspects 14-26, wherein the capillary network comprises continuous capillary channels.

Aspect 28. A process of treating or preventing a disease or condition comprising, administering to a subject in need the composition of any one of aspects 1-13.

Aspect 29. The process of aspect 28, wherein said administering is subcutaneously.

Aspect 30. The process of aspects 28 or 29, wherein said disease or condition is an autoimmune disease, cancer, asthma, an inflammatory disease, an infectious disease, hypercholesterolemia, acute organ rejection, osteoporosis, or Alzheimer's disease.

Aspect 31. Use of any one of aspects 1-13 for the treatment of a disease.

Aspect 32. The use of aspect 31 wherein the disease condition is an autoimmune disease, cancer, asthma, an inflammatory disease, an infectious disease, hypercholesterolemia, acute organ rejection, osteoporosis, or Alzheimer's disease.

While aspects of the disclosure have been illustrated and described, it is not intended that these aspects illustrate and describe all possible forms of the disclosure. Rather, the words used in the specification are words of description rather than limitation, and it is understood that various modifications and substitutions may be made thereto without departing from the spirit and scope of the disclosure.

### PATENT DOCUMENT REFERENCES

| | | |
|---|---|---|
| 6,808,651 B1 | October 2004 | Katagiri, et al. |
| 7,883,664 B2 | February 2011 | G. Elliott; N. Chakraborty. |
| 8,349,252 B2 | January 2013 | G. Elliott; N. Chakraborty. |
| 10,568,318 B2 | February 2020 | P. S. Mohanty, N. Chakraborty. |
| US 2013/0157250 A1 | June 2013 | Gutierrez et al. |
| US 2013/0260452 A1 | October 2013 | Toner et al. |

### NON-PATENT REFERENCES

Chakraborty N, Menze M A, Malsam J, Aksan A, Hand S C, et al. (2011) Cryopreservation of Spin-Dried Mammalian Cells, PLoS ONE 6(9): e24916.
Chakraborty N, Biswas D, Elliott G D (2010) A Simple Mechanistic Way to Increase the Survival of Mammalian Cells During Processing for Dry Storage, Biopreservation and Biobanking, 8 (2), 107-114.

Various modifications of the present disclosure, in addition to those shown and described herein, will be apparent to those skilled in the art of the above description. Such modifications are also intended to fall within the scope of the appended claims.

It is appreciated that all reagents are obtainable by sources known in the art unless otherwise specified.

It is also to be understood that this disclosure is not limited to the specific aspects and methods described herein, as specific components and/or conditions may, of course, vary. Furthermore, the terminology used herein is used only for the purpose of describing particular aspects of the present disclosure and is not intended to be limiting in any way. It will be also understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein. Similarly, as used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference is made in detail to exemplary compositions, aspects and methods of the present disclosure, which constitute the best modes of practicing the disclosure presently known to the inventors. The Figures are not necessarily to scale. However, it is to be understood that the disclosed aspects are merely exemplary of the disclosure that may be embodied in various and alternative forms. Therefore, specific details disclosed herein are not to be interpreted as limiting, but merely as a representative basis for any aspect of the disclosure and/or as a representative basis for teaching one skilled in the art to variously employ the present disclosure.

Patents, publications, and applications mentioned in the specification are indicative of the levels of those skilled in the art to which the disclosure pertains. These patents, publications, and applications are incorporated herein by reference to the same extent as if each individual patent, publication, or application was specifically and individually incorporated herein by reference.

The foregoing description is illustrative of particular embodiments of the disclosure, but is not meant to be a limitation upon the practice thereof. The following claims, including all equivalents thereof, are intended to define the scope of the disclosure.

We claim:

1. A superconcentrated formulation of a bioactive agent comprising: a bioactive agent and an administration solvent, said bioactive agent at a concentration higher than the concentration of said bioactive agent in said administration solvent formulated for subcutaneous administration to a subject.

2. The superconcentrated formulation of claim 1, wherein said solvent is aqueous.

3. The superconcentrated formulation of claim 1, wherein said solvent comprises water and one or more monovalent or divalent salts.

4. The superconcentrated formulation of claim 1, wherein said bioactive agent is present at a concentration above a concentration that said bioactive agent agglomerates in said solvent.

5. The superconcentrated formulation of claim 1, wherein said bioactive agent is present at a concentration in excess of 50 mg/mL, optionally in excess of 90 mg/mL, optionally in excess of 100 mg/mL, optionally in excess of 150 mg/mL.

6. The superconcentrated formulation of any one of claims 1-5, further comprising one or more pharmacologically acceptable excipients.

7. The superconcentrated formulation of any one of claims 1-5, wherein said formulation is in a volume up to 2.5 milliliters.

8. The superconcentrated formulation of any one of claims 1-5 excluding a hyaluronidase.

9. The superconcentrated formulation of any one of claims 1-5, wherein said bioactive agent is an antibody, a protein, a lipid particle, optionally a lipid nanoparticle, or other therapeutic agent.

10. The superconcentrated formulation of claim 9, wherein said bioactive agent is an antibody, optionally a humanized antibody.

11. The superconcentrated formulation of claim 9, wherein said antibody comprises muromonab-CD3, infliximab, rituximab, solanezumab, bapineuzumab, catumaxomab, trastuzumab, cetuximab, omalizumab, adalimumab, bevacizumab, BAN2401, tositumomab, or alemtuzumab.

12. The superconcentrated formulation of claim 9, wherein said bioactive agent is a protein, wherein said protein comprises an interleukin or an interferon, optionally interferon β-1b, Peginterferon alfa-2b, Roferon-A, or aldesleukin.

13. The superconcentrated formulation of any one of claims 1-5, wherein said formulation has a viscosity of 20 cP or less, optionally 12 cP or less, optionally 8 cP or less, optionally 2 cP, or less, optionally 1 cP or less, wherein said viscosity is measured at 20° C. and one atm.

14. A process of preparing the superconcentrated formulation of claim 1, comprising:
a) overlaying a vitrification mixture comprising said bioactive agent and a vitrification medium on a membrane comprising a capillary network, said membrane in a desiccation chamber;
b) lowering atmospheric pressure within the desiccation chamber;
c) providing a heat energy to the vitrification mixture, wherein the heat energy is sufficient to prevent the vitrification mixture from entering a cryogenic state;
d) desiccating the vitrification mixture by capillary action until the vitrification mixture enters a glassy state; and
e) reconstituting said bioactive agent in the administration solvent, whereby the concentration of said bioactive agent in said administration solvent is greater than a concentration of said bioactive agent in said administration solvent when formulated for subcutaneous administration to a subject, optionally greater than or equal to a concentration of said bioactive agent in said vitrification mixture, optionally greater than a concentration of said bioactive agent in said vitrification mixture.

15. The process of claim 14, further comprising repeating steps a-d prior to step e.

16. The process of claim 15, wherein said repeating is 1-3 times.

17. The process of claim 14, wherein the capillary network is provided by contours along a surface of the membrane.

18. The process of claim 14, wherein the capillary network within the desiccation chamber is supported by an underlying solid support substrate.

19. The process of any one of claims 14-18, wherein vitrification of the vitrification mixture occurs in less than 30 minutes, optionally less than 10 minutes.

20. The process of any one of claims 14-18, wherein the heat energy is provided by heating the vitrification mixture.

21. The process of any one of claims 14-18, wherein the atmospheric pressure is lowered to a value of from about 0.9 atm to about 0.005 atm.

22. The process of claim 21, wherein the atmospheric pressure is lowered to about 0.004 atm.

23. The process of any one of claims 14-18, wherein the heat energy provided is sufficient to prevent crystallization of the bioactive agent within the vitrification mixture during vitrification.

24. The process of any one of claims 14-18, wherein the provided heat energy is sufficient to keep the bioactive agent at a temperature of from about 0° C. to about 40° C. during said vitrifying.

25. The process of any one of claims 14-18, wherein said vitrification medium comprises trehalose, glycerol and betine and/or choline.

26. The process of any one of claims 14-18, wherein the capillary network is hydrophilic.

27. The process of any one of claims 14-18, wherein the capillary network comprises continuous capillary channels forming an uninterrupted structure from a first end to a second end.

28. A process of treating or preventing a disease or condition comprising, administering to a subject in need the superconcentrated formulation of any of claims 1-5.

29. The process of claim 28, wherein said administering is subcutaneously.

30. The process of claim 28, wherein said disease or condition is an autoimmune disease, cancer, asthma, an inflammatory disease, an infectious disease, hypercholesterolemia, acute organ rejection, osteoporosis, or Alzheimer's disease.
